(19) United States
(12) Patent Application Publication
(10) Pub. No.: US 2008/0046094 A1
(43) Pub. Date: Feb. 21, 2008

(54) LINEAR TENSION MATERIAL FOR PLASTIC SURGERY

(76) Inventors: Kwang Hee Han, Seoul (KR); Jung Il Lee, Seoul (KR)

Correspondence Address: INTELLECTUAL PROPERTY LAW GROUP LLP, 12 SOUTH FIRST STREET, SUITE 1205, SAN JOSE, CA, 95113, US

(21) Appl. No.: 11/768,106

(22) Filed: Jun. 25, 2007

(30) Foreign Application Priority Data
Jun. 27, 2006 (KR) 10-20060-0057979

(51) Int. Cl. A61F 2/02

(52) U.S. Cl. 623/23.72; 604/22; 604/289

(57) ABSTRACT

The present invention relates to a linear tension material for surgery used to pull skin tissue. An object of the present invention is to provide a linear tension material for plastic surgery which prevents permanent deformation due to excessive elongation and fatigue and of which a portion besides united portions is uniformly and properly united with body tissue. According to an aspect of the present invention, there is provided a linear tension material for plastic surgery which is provided to pull a predetermined site inside skin tissue of the body in a state of tensile load so as to smooth out wrinkles and pull the tissue to tighten a saggy portion, the linear tension material including: a core material in a code shape, which is made of a flexible resin including polyorganosiloxane; and a covering material which covers an outside of the core material to be united with the core material, shrinks together with the core material, and has a fiber textile structure formed by weaving a number of fiber threads.

### CROSS-REFERENCE TO RELATED PATENT APPLICATION

This application claims the benefit of Korean Patent Application No. 10-2006-0057979, filed on Jun. 27, 2006, in the Korean Intellectual Property Office, the disclosure of which is incorporated herein in its entirety by reference.

### BACKGROUND OF THE INVENTION

1. Field of the Invention

The present invention relates to a linear tension material for plastic surgery which is inserted into the skin of the body and pulls skin tissue to smooth out wrinkles, and more particularly, to a linear tension material for plastic surgery capable of maintaining effectiveness of treatment for a long time by presenting plastic deformation caused by load of more than a critical value and fatigue, and improving quality of the treatment by increasing unity with body tissue.

2. Description of the Related Art

Human beings undergo various aging phenomena as they grow older. In particular, wrinkles of the skin are formed because collagen fibers for maintaining elasticity in skin do not function properly and muscles connected to the skin repeatedly move. More particularly, a face has thin skin and is exposed to ultraviolet rays that cause deformation of the collagen fibers for a longer time. In addition, the face has more frequent repeated movements of muscles than other body sites to have various expressions. Therefore, more wrinkles are formed at the face. Since the wrinkles of the face are externally shown, various efforts to remove the wrinkles have been made, and recently, demand for wrinkle removal techniques for various body sites including a neck as well as a face and a technique for tightening a saggy portion increases.

In order to remove wrinkles in each body site, medicines or a technique of peeling off a surface of skin are used. However, as a more efficient method, surgery is used.

As an example of surgery that has been performed, there is a face-lift surgery for pulling skin, incising extra skin, stitching up the incision site, and smoothing out the skin. This surgery has problems in that the surgery has a high risk because a large amount of sites of the body are cut out, scars remain in a treated site, and a long convalescence is required. In addition, as the body tissue is fixed while being pulled, there are disadvantages in that wrinkles due to the aging of the skin are continuously generated, the skin becomes thin due to the pull, and therefore the aging process becomes faster.

As an improved surgery as compared with the aforementioned surgery, surgery for inserting a linear tension material in a code or thread type made of resin into the skin tissue and pulling the skin tissue to tighten a surface of the skin, has been used. This surgery has an additional operation in that collagen fibers and elastic fibers are formed around the linear tension material inserted into the skin and a skin layer becomes thick, so that the surgery can obtain a significant wrinkle removal effect as compared with the past method.

The linear tension material used this surgery is made of various forms and materials. As a representative linear tension material, there is a linear tension material made of polyorganosiloxane with high elasticity in a flexible code type. The linear tension material is inserted into the skin through a needle called an injection needle having a shape of a pipe and is united with a body tissue such as inner skin or muscle to pull a desired body site and smooth out wrinkles. The linear tension material has advantages in that it has good flexibility and elasticity and therefore causes relatively low foreign body sensation after treatment, and more natural plastic surgery is possible.

However, this existing linear tension material has a problem in that due to plastic deformation caused by characteristics of the polyorganosiloxane, a lifting function decreases or is lost as time elapses after the surgery. More specifically, since a conventional polyorganosiloxane has elastic strain of about 300%, when the polyorganosiloxane is elongated above the elastic strain, plastic deformation occurs, and the elongated length of polyorganosiloxane permanently fixed. In addition, when tension is continuously exerted for a long time even under load much less than the elastic strain, permanent deformation occurs due to a creep phenomenon. Therefore, after a predetermined time after the treatment, the treated site cannot maintain a lifted position and hangs down, so that the wrinkle removal function decreases or is lost.

Moreover, since a code surface is smooth, there are problems in that sites besides the united site do not have good unity with the body tissue, and therefore effectiveness of the treatment further decreases. In addition, tension exerted on the treated site is not uniform, so that body deformation such as local depression may occur.

As another conventional linear tension material, a thread made of resin having relatively low strain such as polyester or polypropylene that is used as a conventional suture, has been used. An example thereof is shown in FIG. 1.

A linear tension material 1A shown in FIG. 1 has a number of protrusions 2 at predetermined intervals on a surface so as to increase unity with body tissue. This linear tension material 1A can increase unity with skin over the entire length as well as both ends united with the body tissue, so that a treated site can be more uniformly pulled, and quality of treatment increases.

However, tensile strength of the linear tension material 1A significantly decreases as compared with a linear tension material without the protrusions 2, due to a difference between stress of a portion with the protrusion 2 and a portion without the protrusion 2, so that durability is not good. In addition, a creep phenomenon due to continuous tensile load causes plastic deformation, so that there is a problem in that effectiveness of the treatment is lost in a relatively short time.

In addition, since the linear tension material 1A is made of a material having low elasticity, that is, a relatively stiff material, it causes foreign body sensation when skin tissue moves after the treatment. In particular, a face is treated, there is a problem in that expressions or movements are unnatural.

In addition, there are problems in that the protrusions 2 formed at the surface may offend nervous tissue and cause pain, and the protrusions 2 protrudes and appears from the skin.

### SUMMARY OF THE INVENTION

In order to solve the aforementioned problems, an object of the present invention is to provide a linear tension material for plastic surgery which is not permanently deformed by excessive elongation and fatigue and of which sites as well as a united site are properly united with body tissue.

The present invention are not limited to the aforementioned object, but other objects that are not mentioned will be easily understood by those skilled in the art from the following detailed description.

According to an aspect of the present invention, there is provided a linear tension material for plastic surgery which is provided to pull a predetermined site inside skin tissue of the body in a state of tensile load so as to smooth out wrinkles, the linear tension material including: a core material in a code shape, which is made of a flexible resin including polyorganosiloxane; and a covering material which covers an outside of the core material to be united with the core material, shrinks together with the core material, and has a fiber textile structure formed by weaving a number of fiber threads. The structure of the present invention increases strength for tensile load by uniting the core material and the covering material, so that plastic deformation of the linear tension material for treatment due to excessive elongation and load fatigue is prevented. In addition, cell tissue of a treated site grows between fiber tissue of the covering material, so that unity with body tissue can be uniformly improved.

In the above aspect of the present invention, the core material may have a combined structure having one from among polyorganosiloxane, polyethylene resin, and polypropylene resin, and therefore tensile strength can be further improved.

According to cases, the core material may have a fiber textile structure formed by weaving a number of fiber threads similarly to the covering material. In this case, unity of the linear tension material for the body tissue of the treated site can be further increased.

The fiber threads of the covering material may be made of one of polyester fibers, polyethylene fibers, and polypropylene fibers used for a conventional suture for surgery.

Preferably, the covering material is selected to have strain smaller than strain of the core material as an elongation rate of the core material approaches to a plastic deformation region in consideration of strain due to tensile structure of the fiber threads and strain due to the fiber textile structure. Accordingly, elongation of the core material is suppressed by the covering material 110 before plastic deformation occurs, so that permanent deformation of the linear tension material can be effectively prevented.

In addition, the fiber threads of the covering material may include porous fiber threads having a number of fine notches or fine holes to have a structure for improving unity between the linear tension material and the body tissue.

In addition, at least an end portion of the covering material includes a closure portion which externally extends in a longitudinal direction to be longer than the core material and is formed to be relatively thinner than other portions. The closure portion is used to insert the linear tension material into a needle for surgery inserted into skin tissue and is removed after the linear tension material is united with the skin tissue.

Details according to embodiments are included in the detailed description and drawings.

### BRIEF DESCRIPTION OF THE DRAWINGS

The above and other features and advantages of the present invention will become more apparent by describing in detail exemplary embodiments thereof with reference to the attached drawings in which:

FIG. 1 is a view illustrating a conventional linear tension material for plastic surgery;

FIG. 2 is a perspective view showing a linear tension material for plastic surgery according to an embodiment of the present invention;

FIG. 3 is a cross-sectional view showing a linear tension material for plastic surgery shown in FIG. 2 according to an embodiment of the present invention;

FIG. 4 is a view illustrating various fiber bonding structures of a covering material which is a main element of the present invention; and

FIG. 5 is a treatment status view showing a linear tension material for surgery according to an embodiment of the present invention.

FIG. 6 is another treatment status view showing a linear tension material for surgery according to an embodiment of the present invention.

### DETAILED DESCRIPTION OF THE INVENTION

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art. Like numerals refer to like elements throughout.

Hereinafter, exemplary embodiments of the present invention will be described in detail with reference to the attached drawings.

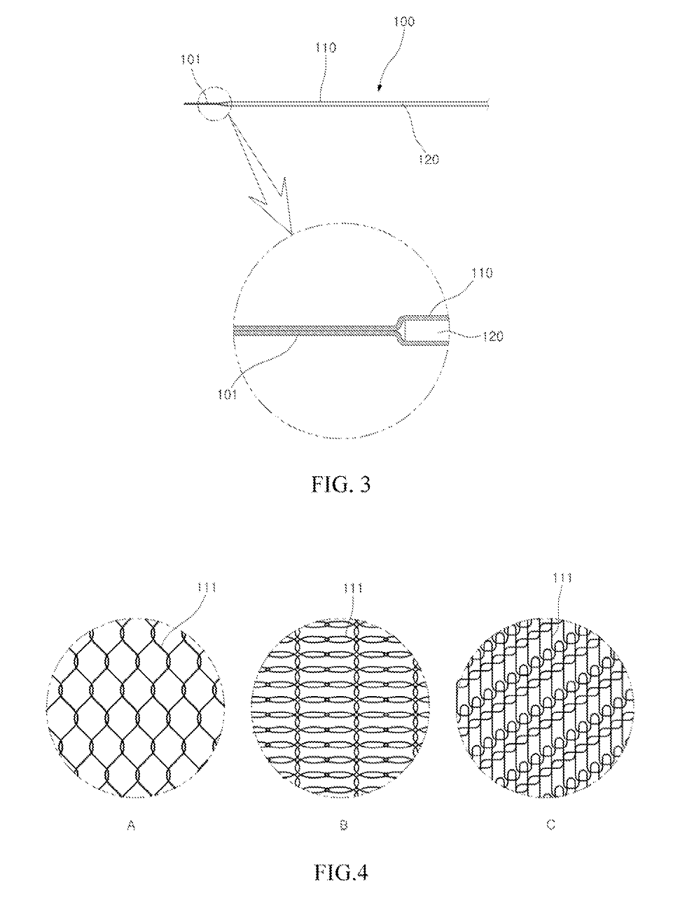

FIG. 2 is a perspective view showing a linear tension material for plastic surgery according to an embodiment of the present invention, and FIG. 3 is a cross-sectional view thereof. Referring to FIGS. 2 and 3, the linear tension material 100 for plastic surgery according to an embodiment of the present invention includes a core material 120 having a predetermined length in a code shape and a covering material 110 for covering the core material 120.

The core material 120 is made of a polymer resin having high flexibility and may be made of a resin conventionally used as a linear tension material for wrinkle removal surgery. In particular, the core material 120 may be made of dimethylsiloxane that has good elasticity and durability and does not offend the body, of derivative or copolymer thereof such as polyorganosiloxane.

The covering material 110 is used to limit an elongation rate of the core material 120 within a proper limit and has a fiber textile structure formed by weaving a number of fiber threads 111. The covering material 110 is adhered to a surface of the core material 120 by shrinkage force due to the textile structure, maintains an adhered state, and elongates or shrinks along with the core material 120.

The fiber threads 111 that form fiber tissue of the covering material 110 may be made of the same material as a suture generally used for surgery, for example, a metal thread including gold, silver, or nickel, polyester, capron, polypropylene, bicryl, polysobe, or the like, and may have various structures including a thread formed with a single fiber as in a conventional fiber thread structure or a thread with a plurality of twisted or braided fibers.

In addition, the fiber thread 111 that forms the covering material 110 may have a structure in which fine notches or fine holes are formed at the surface thereof into a predetermined depth.

In addition, the fiber thread 111 that forms the covering material 110 may be a fiber thread having a surface treated to be smooth. In this case, a capsule is formed around the smoothed surface of the fiber thread inserted into the skin tissue and gradually shrinks, that is, capsulation occurs, so that the saggy portion also shrinks by the capsulation and this generates a force to return to an original state.

The fiber textile structure of the covering material 110 is formed by weaving various types of threads 111 in various forms. The fiber textile structure may be the structure in which fiber threads 111 are weaved in two orthogonal directions as shown in FIG. 2 or a conventional textile structure with various forms that can stretch in a load direction as shown in FIGS. 4A to 4C such as a structure in which the fiber threads 111 are connected in zigzags in a net pattern, a structure in which the fiber threads 111 are connected in a spiral shape 111, and a structure in which the fiber threads 111 are connected to each other while overlap in a hook shape.

In addition, when tension is exerted, an elongation rate of the covering material 110 is determined by strain for tensile load of the fiber thread 111 and strain according to the textile structure. Therefore, in consideration of this, a standard and a structure of the covering material 110 may be selected so that the strain of the core material 120 is smaller than the strain of the covering material 110 as the elongation rate of the core material 120 approaches to a plastic deformation region. This is because the core material 120 should elongate within a deformation limit such that plastic deformation does not occur. For example, according to the current embodiment, plastic deformation occurs when the core material 120 made of polyorganosiloxane elongates at a degree of more than about 300%, so that it is preferable that the core material 120 is easily deformed with elongating until a length of the core material 120 is increased to about 300%, preferably about 200% in consideration of a safety factor, and above the length, the core material 120 has such deformation strength that the core material 120 does not elongate under tensile force.

An end portion of the covering material 110 elongates in a longitudinal direction to be longer than the core material 120 and form a closure portion 101. The closure portion 101 shrinks in a radius direction by shrinkage force due to characteristics of the fiber textile structure and therefore is relatively thinner than other portions.

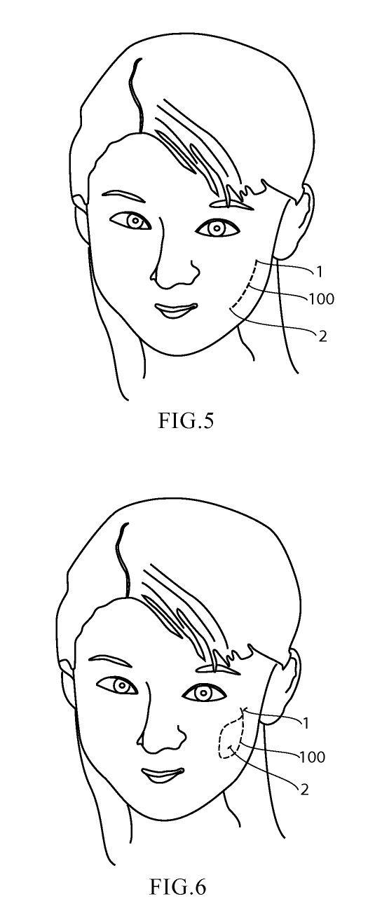

The linear tension material for plastic surgery formed as described above according to an embodiment of the present invention is pushed inside the skin through an inner pipe conduit of a needle for treatment. This can be used for a conventional lift surgery technique, and treatment methods such as a method of estimating a degree of deflection of a treated site before surgery, a method of determining a desired shape for a treated site, a method of determining a lift position of a patient and a united position for supporting the lift position, a treatment method using needles after anesthesia and incision, or the like, that are well known in a related art, may be employed without being limited. An example of a treated state for wrinkle removal of a face is shown in FIG. 5. Referring to FIG. 5, an end portion of the linear tension material 100 is united and fixed to a supporting point 1 such as muscle inside the body and the other end portion is united to a position of subcutaneous tissue to be lifted and is applied with tensile load to lift the treated site.

In addition, as another example of the treated state is shown in FIG. 6. Referring to FIG. 6, the linear tension material 100 is united to enclose a portion 2 to be lifted and is exerted with tension load to lift the treated site.

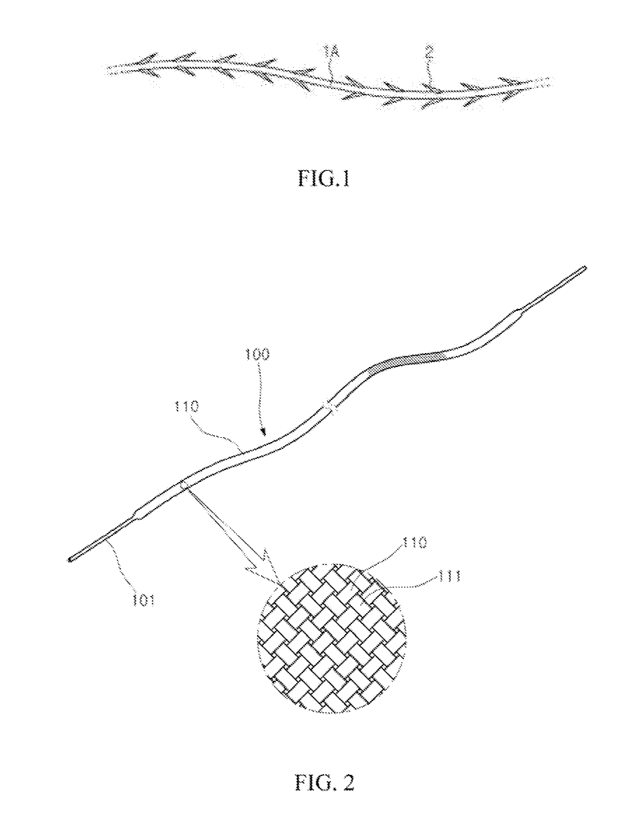

The linear tension material 100 according to an embodiment of the present invention includes the core material 120 and the covering material 110 outside the core material 120 to cooperatively support the tensile load, and therefore has improved tensile strength as compared with a conventional linear tension material including silicon codes and can prevent deflection of the treated site due to the plastic deformation of the core material 120. In addition, while the linear tension material 100 elongates within a predetermined limit, the fiber textile structure of the covering material 110 stretches and binding force for the elongation of the core material 120 is poorly exerted and therefore deformation can easily occur. Therefore, the patient does not have foreign body sensation unlike in the conventional case using the linear tension material shown in FIG. 1 and more natural treated site can be achieved. In addition, cell tissue of the body grows and enters between the fiber structures of the covering material 110, so that unity with skin tissue uniformly increases. Accordingly, local deformation of the treated site is prevented, the entirely uniform lifting can be achieved, and quality of treatment can be improved.

Here, as described above, strain of the covering material 110 is set to be smaller than that of the core material 120 as the core material 120 approaches to a yield region. Therefore, within a limit in which plastic deformation of the core material 120 does not occur, the covering material 110 and the core material 120 electrically elongate together, and above the limit, elongation of the covering material 120 is suppressed by the covering material 110, so that permanent deformation can be effectively prevented.

In addition, both end portions of the covering material 110 are formed with the closure portions 101 which are relatively thin to be easily inserted into a needle for surgery, so that there is an advantage of convenience.

According to the aforementioned embodiment, only the covering material 110 is formed with the fiber tissue. However, according to cases, the core material 120 may have a fiber tissue structure with high elasticity and flexibility as compared with the covering material 110.

In this case, tissue cells in the treated site enter to the core material 120 and unity is improved, so that quality of treatment further increases.

In addition, as described above, the fiber threads 111 of the covering material 110 are formed with porous fiber threads, tissue cells uniformly enter the treated site over the wide part, so that unity between the linear tension material and the skin tissue can be further improved.

According to an embodiment of the present invention, an outside of a flexible core material is united with a covering material with fiber tissue, so that tension strength is increased due to a complex structure and plastic deformation of the core material can be prevented. Simultaneously, elastically flexible deformation can occur within a plastic deformation limit. Therefore, deflection of a treated side due to plastic deformation of the core material can be prevented and treatment for natural shape without foreign body sensation is possible, so that satisfaction of a patient increases.

In addition, somatic cell tissue enters between fiber structures of the covering material, so that tissue of the treated site and a tension material can be strongly and uniformly united, and quality of the treatment can be further improved.

Effectiveness of durability and quality of treatment can be further increased by forming the core material in a fiber structure or forming the covering material with porous fibers.

While the present invention has been particularly shown and described with reference to exemplary embodiments thereof, it will be understood by those skilled in the art that various changes in form and details may be made therein without departing from the spirit and scope of the invention as defined by the appended claims. The exemplary embodiments should be considered in descriptive sense only and not for purposes of limitation. Therefore, the scope of the invention is defined not by the detailed description of the invention but by the appended claims, and all differences within the scope will be construed as being included in the present invention.

What is claimed is:

1. A linear tension material for plastic surgery which is provided to pull a predetermined site inside skin tissue of the body in a state of tensile load so as to smooth out wrinkles and pull the skin tissue to tighten a saggy portion of the skin, the linear tension material comprising:
a core material in a code shape, which is made of a flexible resin including polyorganosiloxane; and
a covering material which covers an outside of the core material to be united with the core material, shrinks together with the core material, and has a fiber textile structure formed by weaving a number of fiber threads.

2. The linear tension material of claim 1, wherein the core material has a combined structure having one from among polyorganosiloxane, polyethylene resin, and polypropylene resin.

3. The linear tension material of claim 1, wherein the core material has a fiber textile structure formed by weaving a number of fiber threads.

4. The linear tension material of claim 1, wherein the fiber textile structure is one of a structure in which fiber threads are weaved in two orthogonal directions, a structure in which the fiber threads are connected in zigzags in a net pattern, a structure in which the fiber threads are connected in a spiral shape, and a structure in which the fiber threads are connected to each other while overlap in a hook shape.

5. The linear tension material of any one of claims 1 to 4, wherein the covering material has strain smaller than strain of the core material as an elongation rate of the core material approaches to a plastic deformation region so that elongation stress of the core material is maintained at a degree within a plastic deformation limit and pulling force is maintained.

6. The linear tension material of any one of claims 1 to 4, wherein the fiber threads of the covering material are made of one of polyester fibers, polyethylene fibers, and polypropylene fibers used for a conventional suture for surgery.

7. The linear tension material of any one of claims 1 to 4, wherein the fiber threads of the covering material include porous fiber threads having a number of fine notches or fine holes.

8. The linear tension material of any one of claims 1 to 4, wherein at least an end portion of the covering material includes a closure portion which externally extends in a longitudinal direction to be longer than the core material and is formed to be relatively thinner than other portions.

9. The linear tension material of any one of claims 1 to 4, wherein the fiber thread that forms the covering material is a fiber thread having a surface treated to be smooth.
